(19) United States
(12) Patent Application Publication
(10) Pub. No.: US 2012/0103790 A1
(43) Pub. Date: May 3, 2012

(54) Continuous Method For Producing Esters Of Aliphatic Carboxylic Acids

(75) Inventors: Matthias Krull, Harxheim (DE); Roman Morschhaeuser, Mainz (DE)

(73) Assignee: CLARIANT FINANCE (BVI) LIMITED, Tortola (VG)

(21) Appl. No.: 13/378,151

(22) PCT Filed: Jun. 9, 2010
(86) PCT No.: PCT/EP2010/003446

§ 371 Date: Dec. 14, 2011

(30) Foreign Application Priority Data
Jun. 30, 2009 (DE) 10 2009 031 053.3

(51) Int. Cl. C07C 51/00

(52) U.S. Cl. 204/157.88; 204/157.87

(57) ABSTRACT

The invention relates to a continuous method for producing aliphatic carbonic acid esters by reacting at least one aliphatic carboxylic acid of formula (I) R¹—COOH (I), wherein R¹ represents hydrogen or an optionally substituted aliphatic hydrocarbon group with 1 to 50 carbon atoms, with at least one alcohol of formula (II) R²—(OH)ₙ (II), wherein R² represents an optionally substituted hydrocarbon group with 1 to 100 C atoms and n is an integer from 1 to 10, in the presence of at least one transesterification catalyst in a reaction tube the longitudinal axis of which extends in the direction of propagation of the microwaves of a monomode microwave applicator, under microwave irradiation to form the ester.

The present invention relates to a continuous method for producing esters of aliphatic carboxylic acids under microwave irradiation on an industrial scale.

Esters are an industrially very important substance group which is used widely and is used, for example, as plasticizer, lubricant and also as a constituent of cosmetics and pharmaceuticals. A proven and often used method for producing esters is the condensation of carboxylic acids with alcohols in the presence of catalysts. In the process, the reaction mixture is usually heated for several hours and the water that is formed is removed. Methods are also known in which the esterification is carried out in a closed system under pressure and high temperatures. For example, WO 2007/126166 discloses a conventionally thermal esterification of fatty acids with alcohols at temperatures of from 200 to 350° C. and pressures of up to 10 bar. During the reaction over several hours, in the course of which the water of reaction which is formed is continuously removed with excess alcohol, however, only an incomplete conversion to the methyl ester is achieved, meaning that a complex work-up and/or further processing of the crude product is required. Another problem of such high-temperature reactions is the corrosivity of the reaction mixtures which, on the one hand, leads to damage of the reaction vessels and, on the other hand, to undesired metal contents in the esters produced in this way.

A more recent approach to the synthesis of esters is the microwave-supported reaction of carboxylic acids and alcohols, as a result of which especially the reaction times required for satisfactory yields are considerably reduced.

Pipus et al. (First European Congress on Chemical Engineering, Firenze, Italy, May 4-7, 1997; AIDIC: Milan, Italy, 1997; pp. 45-48) disclose homogeneously and also heterogeneously catalyzed esterifications of benzoic acid with ethanol in a continuous tubular reactor heated with microwave radiation. At a pressure of 7 atm and a temperature of 140° C., with a residence time in the reactor of 127 seconds, a conversion of 30% is achieved.

WO 03/014272 discloses a method for producing fatty acid methyl esters from triglycerides and methanol using microwave radiation by hydrolysis and esterification and a device for continuously carrying out the method, in which the transesterification takes place in a stirred steel cylinder approximately 120 cm in length, the microwave radiation being coupled into the reaction vessel by means of a multiplicity of magnetrons and waveguides.

US 2005/0274065 discloses processes in which fatty acids are esterified with alcohols in the presence of catalysts and/or under the influence of microwave energy. Here, in one specific embodiment, the reaction material located in a receiver is continually circulated and, in so doing, passed through a stirred container located in a microwave applicator. Only following repeated conveyance through the microwave applicator are high degrees of esterification achieved.

Amore et al. (Macromolecular Rapid Communications, Volume 28 (2007), Issue 4, Pages 473-477) discloses a microwave-assisted method for producing propionic esters, in which the esterification is completed by water removal.

Q. Yang et al. (Synth. Commun. 2008, 38, 4107-4115) describes acid-catalyzed esterifications of various carboxylic acids with alcohols under microwave irradiation. The reactions are carried out at 100° C. on a laboratory scale and lead to high conversions.

The scale-up of such microwave-supported reactions from the laboratory to an industrial scale and thus the development of plants which are suitable for a production of several tons, for example several tens, several hundreds or several thousands of tons, per year with space-time yields of interest for industrial-scale applications has, however, not been realized to date. One reason for this is the penetration depth of microwaves into the reaction material, which is usually limited to a few millimeters to a few centimeters, which limits especially reactions carried out in batch processes to small vessels, or leads to very long reaction times in stirred reactors. Tight limits are placed on an increase in the field strength, which is desirable for the irradiation of large amounts of substance with microwaves, especially in the multimode devices used preferentially to date for scale-up of chemical reactions as a result of the discharge processes and plasma formation which then arise. Furthermore, the inhomogeneity of the microwave field, which leads to local overheating of the reaction material in multimode microwave devices and is caused by more or less uncontrolled reflections of the microwaves injected into the microwave oven at the walls thereof and the reaction mixture, presents problems in the scale-up. Furthermore, the microwave absorption coefficient of the reaction mixture, which often changes during the reaction, presents difficulties with regard to a safe and reproducible reaction regime.

WO 90/03840 discloses a continuous method for carrying out various chemical reactions, such as, for example, esterifications, in a continuous laboratory microwave reactor. However, the achieved yields and also the reaction volume of 24 ml of the microwave operated in multimode, do not permit upscaling to the industrial sector. The efficiency of this method with regard to the microwave absorption of the reaction material is low on account of the microwave energy being more or less homogeneously distributed over the applicator space in multimode microwave applicators and not focused on the tube coil. A significant increase in the microwave power injected can lead to undesired plasma discharges or to so-called thermal runaway effects. Furthermore, the spatial inhomogeneities of the microwave field in the applicator space, which are referred to as hot-spots and change over time, make a safe and reproducible reaction regime on a large scale impossible.

Also known are monomode or single-mode microwave applicators which use a single wave mode which propagates in only one three-dimensional direction and is focused onto the reaction vessel by waveguides of exact dimensions. Although these instruments do allow relatively high local field strengths, on account of the geometric requirements (e.g. the intensity of the electrical field is at its greatest at its wave crests and approaches zero at the nodes), they have hitherto been restricted to small reaction volumes (50 ml) on the laboratory scale.

For example, Chemat et al. (J. Microwave Power and Electromagnetic Energy 1998, 33, 88-94) disclose various continuous esterifications in a monomode microwave cavity, where the microwave guide is perpendicular to the reaction tube. Here, accelerated conversions are observed in the case of heterogeneously catalyzed esterifications. The volume of only 20 ml available for the microwave irradiation, however, requires that the reactants be repeatedly conveyed through the irradiation zone in order to achieve interesting yields. A significant increase in the cross section of the reaction tube is not possible on account of the geometry of the applicator and is also not suitable for the upscaling on account of the low penetration depth of microwaves.

Esveld et al. (Chem. Eng. Technol. 23 (2000), 429-435) disclose a continuous method for producing wax esters, in which fatty alcohol and fatty acid are esterified without solvent in the presence of montmorillonite. The reaction mixture is conveyed on a conveyor belt through a microwave cavity, the condensation being completed by extensive removal of the water of reaction which is formed. This method can naturally only be used for high-boiling alcohols and acids.

A method was therefore sought for producing esters of aliphatic carboxylic acids, in which aliphatic carboxylic acid and alcohol can also be converted to the ester on an industrial scale under microwave irradiation. In this connection, the aim was to achieve the highest possible, i.e. up to quantitative, conversion rates and yields. Furthermore, the method should permit as energy-saving a production of the esters as possible, i.e. the microwave power used should be absorbed as quantitatively as possible by the reaction material and the method should thus offer a high energetic efficiency. In the process, only minor amounts of by-products, if any, should be produced. The esters should also have the lowest possible metal content and a low intrinsic coloration. Moreover, the method should ensure a safe and reproducible reaction regime.

Surprisingly, it has been found that esters of aliphatic carboxylic acids can be produced in industrially relevant amounts by direct reaction of aliphatic carboxylic acids with alcohols in a continuous method by only briefly heating by means of irradiation with microwaves in a reaction tube, the longitudinal axis of which is in the direction of propagation of the microwaves of a monomode microwave applicator. Here, the microwave energy injected into the microwave applicator is virtually quantitatively absorbed by the reaction material. The method according to the invention additionally has high safety during the procedure and offers high reproducibility of the reaction conditions established. The esters produced by the method according to the invention exhibit a high purity and low intrinsic coloration not obtainable in comparison to by conventional production methods without additional method steps.

The invention provides a continuous method for producing carboxylic esters, in which at least one aliphatic carboxylic acid of the formula (I)

R¹—COOH (I)

in which R¹ is hydrogen or an optionally substituted aliphatic hydrocarbon radical having 1 to 50 carbon atoms,
is reacted with at least one alcohol of the formula (II)

R²—(OH)ₙ (II)

in which

R² is an optionally substituted hydrocarbon radical having 1 to 100 carbon atoms and

n is a number from 1 to 10,

in the presence of at least one esterification catalyst with microwave irradiation in a reaction tube, the longitudinal axis of which extends in the direction of propagation of the microwaves of a monomode microwave applicator, to give the ester.

Suitable aliphatic carboxylic acids of the formula (I) are generally compounds which have at least one carboxyl group on an optionally substituted aliphatic hydrocarbon radical having 1 to 50 carbon atoms, and also formic acid. In a preferred embodiment, the aliphatic hydrocarbon radical is an unsubstituted alkyl or alkenyl radical. In a further preferred embodiment, the aliphatic hydrocarbon radical carries one or more, such as, for example two, three, four or more, further substituents. Suitable substituents are, for example, halogen atoms, halogenated alkyl radicals, hydroxyl, C₁-C₅-alkoxy and for example methoxy, poly(C₁-C₅-alkoxy), poly(C₁-C₅-alkoxy)alkyl, carboxyl, ester, amide, cyano, nitrile, nitro and/or aryl groups having 5 to 20 carbon atoms, such as, for example, phenyl groups, with the proviso that these are stable under the reaction conditions and do not enter into any secondary reactions such as, for example, elimination reactions. The C₅-C₂₀-aryl groups can for their part in turn carry substituents such as, for example, halogen atoms, halogenated alkyl radicals, C₁-C₂₀-alkyl, C₂-C₂₀-alkenyl, C₁-C₅-alkoxy such as, for example, methoxy, ester, amide, cyano, nitrile and/or nitro groups. The aryl groups can contain one or more heteroatoms, such as, for example, nitrogen, oxygen and/or sulfur, but not more heteroatoms than carbon atoms. The aliphatic hydrocarbon radical carries at most as many substituents as it has valences. In a specific embodiment, the aliphatic hydrocarbon radical R¹ carries further carboxyl groups. The process according to the invention is thus likewise suitable for the esterification of polycarboxylic acids having, for example, two, three, four or more carboxyl groups. In this connection, the carboxyl groups can be esterified completely or only partially. The degree of esterification can be adjusted, for example, via the stoichiometry between carboxylic acid and alcohol in the reaction mixture.

According to the invention, particular preference is given to carboxylic acids (I) which carry an aliphatic hydrocarbon radical having 1 to 30 carbon atoms and in particular having 2 to 24 carbon atoms, such as, for example, having 3 to 20 carbon atoms. They can be of natural or synthetic origin. The aliphatic hydrocarbon radical can also contain heteroatoms such as, for example, oxygen, nitrogen, phosphorus and/or sulfur, but preferably not more than one heteroatom per 3 carbon atoms.

The aliphatic hydrocarbon radicals can be linear, branched or cyclic. The carboxyl group can be bonded to a primary, secondary or tertiary carbon atom. Preferably, it is bonded to a primary carbon atom. The hydrocarbon radicals can be saturated or, if their hydrocarbon radical R¹ includes at least 2 carbon atoms, also unsaturated. Unsaturated hydrocarbon radicals preferably contain one or more C═C double bonds and particularly preferably one, two or three C═C double bonds. The process according to the invention has thus proven particularly useful for producing esters of polyunsaturated carboxylic acids since the double bonds of the unsaturated carboxylic acids are not attacked under the reaction conditions of the process according to the invention. Preferred cyclic aliphatic hydrocarbon radicals have at least one ring with four, five, six, seven, eight or more ring atoms.

In a preferred embodiment, R¹ is a saturated alkyl radical having 1, 2, 3 or 4 carbon atoms. This may be linear or, in the case of 4 carbon atoms, also branched. The carboxyl group can be bonded to a primary, secondary or, as in the case of pivalic acid, tertiary carbon atom. In a particularly preferred embodiment, the alkyl radical is an unsubstituted alkyl radical. In a further particularly preferred embodiment, the alkyl radical carries one to nine, preferably one to five, such as, for example, two, three or four further substituents. Preferred further substituents are carboxyl groups, hydroxyl groups, and also optionally substituted C₅-C₂₀-aryl radicals.

In a further preferred embodiment, the carboxylic acid (I) is an ethylenically unsaturated carboxylic acid. Here, R¹ is an optionally substituted alkenyl group having 2 to 4 carbon atoms. Ethylenically unsaturated carboxylic acids are understood here as meaning those carboxylic acids which have a C═C double bond conjugated to the carboxyl group. In a preferred embodiment, the alkenyl radical is an unsubstituted alkenyl radical. Particularly preferably, R¹ is an alkenyl radical having 2 or 3 carbon atoms. In a further preferred embodiment, the alkenyl radical carries one or more, such as, for example, two, three or more, further substituents. However, the alkenyl radical carries at most as many substituents as it has valences. In a preferred embodiment, the alkenyl radical R¹ carries, as further substituents, a carboxyl group or an optionally substituted C₅-C₂₀-aryl group. The process according to the invention is thus likewise suitable for reacting ethylenically unsaturated dicarboxylic acids.

In a further preferred embodiment, the carboxylic acid (I) is a fatty acid. In this connection, R¹ is an optionally substituted aliphatic hydrocarbon radical having 5 to 50 carbon atoms. Particular preference is given here to fatty acids which carry an aliphatic hydrocarbon radical having 6 to 30 carbon atoms and in particular having 7 to 26 carbon atoms, such as, for example, having 8 to 22 carbon atoms. In a preferred embodiment, the hydrocarbon radical of the fatty acid is an unsubstituted alkyl or alkenyl radical. In a further preferred embodiment, the hydrocarbon radical of the fatty acid carries one or more, such as, for example, two, three, four or more, further substituents.

Carboxylic acids suitable for the esterification according to the process of the invention are, for example, formic acid, acetic acid, propionic acid, butyric acid, isobutyric acid, pentanoic acid, isopentanoic acid, pivalic acid, acrylic acid, methacrylic acid, crotonic acid, 2,2-dimethylacrylic acid, maleic acid, fumaric acid, itaconic acid, cinnamic acid and methoxycinnamic acid, succinic acid, butanetetracarboxylic acid, phenylacetic acid, (2-bromophenyl)acetic acid, (methoxyphenyl)acetic acid, (dimethoxyphenyl)acetic acid, 2-phenylpropionic acid, 3-phenylpropionic acid, 3-(4-hydroxyphenyl)propionic acid, 4-hydroxyphenoxyacetic acid, indole acetic acid, hexanoic acid, cyclohexanoic acid, heptanoic acid, octanoic acid, nonanoic acid, neononanoic acid, decanoic acid, neodecanoic acid, undecanoic acid, neoundecanoic acid, dodecanoic acid, tridecanoic acid, isotridecanoic acid, tetradecanoic acid, 12-methyltridecanoic acid, pentadecanoic acid, 13-methyltetradecanoic acid, 12-methyltetradecanoic acid, hexadecanoic acid, 14-methylpentadecanoic acid, heptadecanoic acid, 15-methylhexadecanoic acid, 14-methylhexadecanoic acid, octadecanoic acid, isooctadecanoic acid, eicosanoic acid, docosanoic acid and tetracosanoic acid, myristoleic, palmitoleic, hexadecadienoic, delta-9-cis-heptadecenoic acid, oleic, petroselic, vaccenic, linoleic, linolenic, gadoleic, gondoic, eicosadienoic, arachidonic, cetoleic, erucic, docosadienoic and tetracosenoic acid, 2-hydroxypropionic acid, 3-hydroxypropionic acid, 2-hydroxybutyric acid, 3-hydroxybutyric acid, 4-hydroxybutyric acid, 2-hydroxy-2-methylpropionic acid, 4-hydroxypentanoic acid, 5-hydroxypentanoic acid, 2,2-dimethyl-3-hydroxypropionic acid, 5-hydroxyhexanoic acid, 2-hydroxyoctanoic acid, 2-hydroxytetradecanoic acid, 15-hydroxypentadecanoic acid, 16-hydroxyhexadecanoic acid and 12-hydroxystearic acid, dodecenylsuccinic acid, octadecenylsuccinic acid, hydroxysuccinic acid, citric acid and dimer fatty acids which can be produced from unsaturated fatty acids, and also mixtures thereof. Also of suitability are carboxylic acid mixtures obtained from natural fats and oils, such as, for example, cotton seed, coconut, peanut, safflower, corn, palm kernel, rapeseed, olive, mustard seed, soya, sunflower oil and also tallow, bone and fish oil. Likewise suitable as carboxylic acids or carboxylic acid mixtures for the process according to the invention are tall oil fatty acid and also resin and naphthenic acids.

Lower aliphatic carboxylic acids having 1 to 4 carbon atoms that are particularly preferred according to the invention are formic acid, acetic acid and propionic acid, 2-hydroxypropionic acid, and also phenylacetic acid and its derivatives substituted on the aryl radical. Particularly preferred ethylenically unsaturated carboxylic acids are acrylic acid and methacrylic acid. Particularly preferred fatty acids are rapeseed oil fatty acid, coconut fatty acid, stearic acid, tallow fatty acid and tall oil fatty acid.

In a preferred embodiment, R² is an aliphatic radical. This has preferably 1 to 24, particularly preferably 2 to 18 and specifically 3 to 6, carbon atoms. The aliphatic radical can be linear, branched or cyclic. It can also be saturated or, if it has at least three carbon atoms, unsaturated, it is preferably saturated. The hydrocarbon radical can carry substituents such as, for example, halogen atoms, halogenated alkyl radicals, hydroxyl, C₁-C₅-alkoxyalkyl, carboxyl, cyano, nitrile, nitro and/or C₅-C₂₀-aryl groups, such as, for example, phenyl radicals. The C₅-C₂₀-aryl radicals can for their part be optionally substituted with halogen atoms, halogenated alkyl radicals, hydroxyl, C₁-C₂₀-alkyl, C₂-C₂₀-alkenyl, C₁-C₅-alkoxy groups, such as, for example, methoxy, ester, amide, cyano, nitrile and/or nitro groups. Particularly preferred aliphatic radicals R² are methyl, ethyl, n-propyl, isopropyl, n-butyl, isobutyl and tert-butyl, n-hexyl, cyclohexyl, n-octyl, n-decyl, n-dodecyl, tridecyl, isotridecyl, tetradecyl, hexadecyl, octadecyl and methylphenyl.

In a further preferred embodiment, R² is an optionally substituted C₆-C₁₂-aryl group or an optionally substituted heteroaromatic group having 5 to 12 ring members. Preferred heteroatoms are oxygen, nitrogen and sulfur. Further rings can be fused onto the C₆-C₁₂-aryl group carrying at least one hydroxyl group or the heteroaromatic group having 5 to 12 ring members. The aryl or heteroaromatic group can thus be mono- or polycyclic. Examples of suitable substituents are halogen atoms, halogenated alkyl radicals and also alkyl, alkenyl, hydroxy, hydroxyalkyl, alkoxy, ester, amide, nitrile and nitro groups.

In a specific embodiment, the radical R² carries one or more, such as, for example, two, three, four, five, six or more, further hydroxyl groups, but not more hydroxyl groups than the radical R² has carbon atoms or than the aryl group has valences. The hydroxyl groups can be bonded to adjacent carbon atoms or else to further removed carbon atoms of the hydrocarbon radical, but at most one OH group per carbon atom. Thus, the method according to the invention is also suitable for the esterification of polyols such as, for example, ethylene glycol, 1,2-propanediol, 1,3-propanediol, neopentyl glycol, glycerol, sorbitol, pentaerythritol, fructose and glucose. The esterification can be conducted here to full esters or else partial esters. The degree of esterification can be controlled here for example via the stoichiometry between carboxylic acid and alcohol in the reaction mixture.

In a further preferred embodiment, R² is an alkyl radical interrupted with heteroatoms. Particularly preferred heteroatoms are oxygen and nitrogen. If the radical R² contains nitrogen atoms, then these nitrogen atoms carry no acidic protons though.

Thus, R² is preferably radicals of the formula (III)

—(R⁴—O)ₙR⁵ (III)

in which

R⁴ is an alkylene group having 2 to 18 carbon atoms, preferably having 2 to 12 and in particular 2 to 4, carbon atoms, such as, for example, ethylene, propylene, butylene or mixtures thereof,

R⁵ is hydrogen or a hydrocarbon radical having 1 to 24 carbon atoms or a group of the formula —R⁴—NR¹⁰R¹¹,

n is a number between 1 and 500, preferably between 2 and 200 and in particular between 3 and 50, such as, for example, between 4 and 20, and

R¹⁰, R¹¹ independently of one another, are an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, where the polyoxyalkylene units are derived from alkylene oxide units having 2 to 6 carbon atoms, or R¹⁰ and R¹¹ together with the nitrogen atom to which they are bonded are a ring having 4, 5, 6 or more ring members.

Examples of suitable alcohols are methanol, ethanol, n-propanol, isopropanol, n-butanol, isobutanol, tert-butanol, pentanol, neopentanol, n-hexanol, isohexanol, cyclohexanol, heptanol, octanol, decanol, dodecanol, tetradecanol, hexadecanol, octadecanol, eicosanol, ethylene glycol, 2-methoxyethanol, propylene glycol, diethylene glycol, triethylene glycol, polyethylene glycol, polypropylene glycol, triethanolamine, N,N-dimethylethanolamine, N,N-diethylethanolamine, phenol, naphthol and mixtures thereof. Also of suitability are fatty alcohol mixtures obtained from natural raw materials, such as, for example, coconut fatty alcohol, palm kernel fatty alcohol and tallow fatty alcohol.

The method is particularly suitable for producing ethyl formate, methyl acetate, ethyl acetate, ethyl propionate, stearyl stearate and rapeseed oil fatty acid methyl ester.

In cases where the carboxylic acid (I) contains two or more carboxyl groups and the alcohol (II) contains two or more hydroxyl groups and/or both reactants, as in the case of hydroxycarboxylic acids, in each case carry at least one carboxyl group and at least one hydroxyl group, it also being possible for the reactants (I) and (II) to be identical, oligomers and polymers can also be produced by the method according to the invention. For example, oligomers and polymers of lactic acid can thus be produced by the method according to the invention. In the case of such polycondensations, the viscosity of the reaction mixture, which increases during the microwave irradiation, is to be taken into consideration when designing the apparatus.

In the method according to the invention, aliphatic carboxylic acid (I) and alcohol (II) can be reacted with one another in any desired ratios. Preferably, the reaction between carboxylic acid and alcohol takes place with molar ratios of from 20:1 to 1:20, preferably from 10:1 to 1:10 and specifically from 3:1 to 1:3, such as, for example, from 1.5:1 to 1:1.5, in each case based on the mole equivalents of carboxyl groups and hydroxyl groups. In a specific embodiment, carboxylic acid and alcohol are used in equimolar amounts. If the aliphatic carboxylic acid (I) carries one or more hydroxyl groups, the reaction preferably takes place with at least equimolar fractions of alcohol (II), particularly preferably in the ratio of aliphatic carboxylic acid (I) to alcohol (II) of from 1:1.01 to 1:50, specifically in the ratio 1:1.5 to 1:20, such as, for example, 1:2 to 1:10.

In many cases, it has proven to be advantageous to work with an excess of alcohol, i.e. molar ratios of hydroxyl groups to carboxyl groups of at least 1.01:1.00 and in particular between 50:1 and 1.02:1, such as, for example, between 10:1 and 1.1:1. Here, the carboxyl groups are converted virtually quantitatively to the ester. This method is particularly advantageous if the alcohol used is readily volatile. Readily volatile means here that the alcohol has a boiling point at atmospheric pressure of preferably below 200° C. and particularly preferably below 160° C., such as, for example, below 100° C., and can thus be separated off from the ester by distillation.

The esterifications are carried out in the method according to the invention in the presence of homogeneous catalysts, heterogeneous catalysts or mixtures thereof. Both acidic and alkali catalysts are suitable here. Esterification catalysts preferred according to the invention are acidic inorganic, organometallic or organic catalysts and mixtures of two or more of these catalysts.

Acidic inorganic catalysts within the context of the present invention include, for example, sulfuric acid, phosphoric acid, phosphonic acid, hypophosphorous acid, aluminum sulfate hydrate, alum, acidic silica gel and acidic aluminum hydroxide. It is also possible to use, for example, aluminum compounds of the general formula Al(OR¹⁵)₃ and titanates of the general formula Ti(OR¹⁵)₄ as acidic inorganic catalysts, where the radicals R¹⁵ can in each case be identical or different and, independently of one another, are selected from C₁-C₁₀-alkyl radicals, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, sec-hexyl, n-heptyl, n-octyl, 2-ethylhexy, n-nonyl or n-decyl, C₃-C₁₂-cycloalkyl radicals, for example cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl. The radicals R¹⁵ in Al(OR¹⁵)₃ or Ti(OR¹⁵)₄ are preferably in each case identical and selected from isopropyl, butyl and 2ethylhexyl.

Preferred acidic organometallic catalysts are, for example, selected from dialkyltin oxides (R¹⁵)₂SnO, where R¹⁵ is as defined above. A particularly preferred representative of acidic organometallic catalysts is di-n-butyltin oxide, which is commercially available as Oxo-tin or as Fascat® grades.

Preferred acidic organic catalysts are acidic organic compounds having, for example, phosphate groups, sulfonic acid groups, sulfate groups or phosphonic acid groups. Particularly preferred sulfonic acids contain at least one sulfonic acid group and at least one saturated or unsaturated, linear, branched and/or cyclic hydrocarbon radical having 1 to 40 carbon atoms and preferably having 3 to 24 carbon atoms. Particular preference is given to aromatic sulfonic acids and specifically alkylaromatic monosulfonic acids having one or more C₂-C₂₈-alkyl radicals and in particular those having C₃-C₂₂-alkyl radicals. Suitable examples are methanesulfonic acid, butanesulfonic acid, benzenesulfonic acid, p-toluenesulfonic acid, xylenesulfonic acid, 2-mesitylenesulfonic acid, 4-ethylbenzenesulfonic acid, isopropylbenzenesulfonic acid, 4-butylbenzenesulfonic acid, 4-octylbenzene-sulfonic acid; dodecylbenzenesulfonic acid, didodecylbenzenesulfonic acid, naphthalenesulfonic acid. It is also possible to use acidic ion exchangers as acidic organic catalysts, for example sulfone-group-carrying poly(styrene) resins crosslinked with about 2 mol % of divinylbenzene.

Of particular preference for carrying out the method according to the invention are boric acid, phosphoric acid, polyphosphoric acid and polystyrenesulfonic acids. Particular preference is given to titanates of the general formula Ti(OR¹⁵)₄ and specifically titanium tetrabutylate and titanium tetraisopropylate.

If the use of acidic inorganic, organometallic or organic catalysts is desired, then according to the invention, 0.01 to 10% by weight, preferably 0.02 to 2% by weight, of catalyst is used.

In a further preferred embodiment, the microwave irradiation is carried out in the presence of acidic solid catalysts. Heterogeneous catalysts of this type can be suspended in the reaction mixture and pumped through the reaction tube together with the reaction mixture. In a particularly preferred embodiment, the reaction mixture, optionally admixed with solvent, is passed over a fixed-bed catalyst located in the reaction tube and, in so doing, subjected to microwave radiation. Suitable solid catalysts are, for example, zeolites, silica gel, montmorillonite and (partially) crosslinked polystyrenesulfonic acid, which may optionally be impregnated with catalytically active metal salts. Suitable acidic ion exchangers based on polystyrenesulfonic acids, which can be used as solid-phase catalysts, are obtainable, for example, from Rohm & Haas under the trade name Amberlyst®.

The production according to the invention of the esters takes place by mixing carboxylic acid, alcohol and catalyst and subsequent irradiation of the mixture with microwaves in a reaction tube, the longitudinal axis of which is in the direction of propagation of the microwaves in a monomode microwave applicator.

The irradiation of the reaction mixture with microwaves preferably takes place in a largely microwave-transparent reaction tube located within a hollow conductor connected to a microwave generator. The reaction tube is preferably aligned axially with the central axis of symmetry of the hollow conductor.

The hollow conductor functioning as microwave applicator is preferably configured as a cavity resonator. Further preferably, the microwaves not absorbed in the hollow conductor are reflected at its end. Preferably, the length of the cavity resonator is dimensioned such that a stationary wave is formed therein. By configuring the microwave applicator as a resonator of the reflection type, a local increase in the electrical field strength at the same power supplied by the generator and increased energy exploitation are achieved.

The cavity resonator is preferably operated in the E₀₁ₙ mode, where n is an integer and indicates the number of field maxima of the microwave along the central axis of symmetry of the resonator. In this operation, the electrical field is directed in the direction of the central axis of symmetry of the cavity resonator. It has a maximum in the region of the central axis of symmetry and decreases to the value zero toward the outer surface. This field configuration is rotationally symmetric about the central axis of symmetry. By using a cavity resonator with a length in which n is an integer, the formation of a stationary wave is facilitated. According to the desired flow rate of the reaction material through the reaction tube, the required temperature and the required residence time in the resonator, the length of the resonator is selected relative to the wavelength of the microwave radiation used. Preferably, n is an integer from 1 to 200, particularly preferably from 2 to 100, in particular from 4 to 50, and specifically from 3 to 20, such as, for example, three, four, five, six, seven, eight, nine or ten.

The E₀₁ₙ mode of the cavity resonator is also referred to as TM₀₁ₙ mode, see for example K. Lange, K. H. Löcherer, Taschenbuch der Hochfrequenztechnik [Pocket book of high-frequency technology], volume 2, page K21 ff.

The injection of the microwave energy into the hollow conductors functioning as microwave applicator can take place via suitably dimensioned holes or slits. In a particularly preferred embodiment according to the invention, the irradiation of the reaction mixture with microwaves takes place in a reaction tube which is in a hollow conductor with a coaxial transition of the microwaves. Microwave devices particularly preferred for this method are constructed from a cavity resonator, a coupling device for coupling a microwave field into the cavity resonator and with in each case one orifice on two opposite end walls for passage of the reaction tube through the resonator. The microwaves are preferably coupled into the cavity resonator via a coupling pin which projects into the cavity resonator. Preferably, the coupling pin is configured as a preferably metallic inner conductor tube which functions as a coupling antenna. In a particularly preferred embodiment, this coupling pin projects through one of the end orifices into the cavity resonator. The reaction tube particularly preferably adjoins the inner conductor tube of the coaxial transition and is specifically conducted through the cavity thereof into the cavity resonator. The reaction tube is preferably aligned axially with a central axis of symmetry of the cavity resonator. For this, the cavity resonator preferably has in each case one central orifice on two opposite end walls for passage of the reaction tube.

The feeding-in of the microwaves into the coupling pin or into the inner conductor tube functioning as a coupling antenna can take place, for example, by means of a coaxial connecting line. In a preferred embodiment, the microwave field is supplied to the resonator via a hollow conductor, in which case the end of the coupling pin projecting out of the cavity resonator is conducted into an orifice, which is located in the wall of the hollow conductor, into the hollow conductor, and takes microwave energy from the hollow conductor and couples it into the resonator.

In a specific embodiment, the irradiation of the reaction mixture with microwaves takes place in a microwave-transparent reaction tube which is axially symmetrical within a E₀₁ₙ round hollow conductor with a coaxial transition of the microwaves. In this case, the reaction tube is conducted through the cavity of an inner conductor tube functioning as coupling antenna into the cavity resonator. In a further preferred embodiment, the irradiation of the reaction mixture with microwaves takes place in a microwave-transparent reaction tube which is conducted through a E₀₁ₙ cavity resonator with axial feeding-in of the microwaves, where the length of the cavity resonator is dimensioned such that n=2 or more field maxima of the microwave are formed. In a further preferred embodiment, the irradiation of the reaction mixture with microwaves takes place in a microwave-transparent reaction tube which is conducted through a E₀₁ₙ cavity resonator with axial feeding-in of the microwaves, where the length of the cavity resonator is dimensioned such that a stationary wave where n=2 or more field maxima of the microwave is formed. In a further preferred embodiment, the irradiation of the reaction mixture with microwaves takes place in a microwave-transparent reaction tube which is axially symmetric within a circular cylindrical E₀₁ₙ cavity resonator with a coaxial transition of the microwaves, where the length of the cavity resonator is dimensioned such that n=2 or more field maxima of the microwave are formed. In a further preferred embodiment, the irradiation of the reaction mixture with microwaves takes place in a microwave-transparent reaction tube which is axially symmetric within a circular cylindrical E₀₁ₙ cavity resonator with a coaxial transition of the microwaves, where the length of the cavity resonator is dimensioned such that a stationary wave where n=2 or more field maxima of the microwave is formed.

Microwave generators, such as, for example, the magnetron, the klystron and the gyrotron are known to the person skilled in the art.

The reaction tubes used to carry out the method according to the invention are preferably manufactured from largely microwave-transparent, high-melting material. Particular preference is given to using nonmetallic reaction tubes. Largely microwave-transparent is understood here as meaning materials which absorb as little microwave energy as possible and convert it to heat. A measure used for the ability of a substance to absorb microwave energy and convert it to heat is often the dielectric loss factor tan δ=ε″/ε′. The dielectric loss factor tan δ is defined as the ratio of dielectric loss ε″ and dielectric constant ε′. Examples of tan δ values of various materials are given, for example, in D. Bogdal, Microwave-assisted Organic Synthesis, Elsevier 2005. For reaction tubes suitable according to the invention, materials with tan δ values measured at 2.45 GHz and 25° C. of less than 0.01, in particular less than 0.005 and specifically less than 0.001 are preferred. Suitable preferred microwave-transparent and thermally stable materials are primarily mineral-based materials such as, for example, quartz, aluminum oxide, sapphire, zirconium oxide, silicon nitride and the like. Thermally stable plastics such as, in particular, fluoropolymers, such as, for example, Teflon, and industrial plastics such as polypropylene, or polyaryl ether ketones, such as, for example, glass fiber-reinforced polyether ether ketone (PEEK), are also suitable as tube materials. In order to withstand the temperature conditions during the reaction, minerals, such as quartz or aluminum oxide, coated with these plastics have in particular proven to be useful as reactor materials.

Reaction tubes particularly suitable for the method according to the invention have an internal diameter of one millimeter to ca. 50 cm, in particular between 2 mm and 35 cm and specifically between 5 mm and 15 cm, such as, for example, between 10 mm and 7 cm. Reaction tubes are understood here as meaning vessels whose length to diameter ratio is greater than 5, preferably between 10 and 100 000, particularly preferably between 20 and 10 000, such as, for example, between 30 and 1000. The length of the reaction tube is understood here as meaning the length of the reaction tube on which the microwave irradiation takes place. Baffles and/or other mixing elements can be incorporated into the reaction tube.

E₀₁ cavity resonators particularly suitable for the method according to the invention preferably have a diameter which corresponds to at least half the wavelength of the microwave radiation used. Preferably, the diameter of the cavity resonator is 1.0 to 10 times, more preferably 1.1 to 5 times and especially 2.1 to 2.6 times, half the wavelength of the microwave radiation used. Preferably, the E₀₁ cavity resonator has a round cross section, which is also referred to as E₀₁ round hollow conductor. It particularly preferably has a cylindrical shape and specifically a circular cylindrical shape.

The reaction tube is usually provided at the inlet with a metering pump and a manometer, and at the outlet with a pressure-retaining device and a heat exchanger. This makes possible reactions within a very wide pressure and temperature range.

The production of the reaction mixture consisting of carboxylic acid, alcohol and catalyst can be carried out continuously, discontinuously or else in semi-batchwise processes. Thus, the production of the reaction mixture can be carried out in an upstream (semi)-batchwise process, such as, for example, in a stirred vessel. in a preferred embodiment, the starting materials carboxylic acid and alcohol, and also the catalyst, independently of one another optionally diluted with solvent, are only mixed shortly before being introduced into the reaction tube. The catalyst can be added to the reaction mixture as it is or as a mixture with one of the starting materials. For example, it has proven particularly useful to undertake the mixing of carboxylic acid, alcohol and catalyst in a mixing zone, from which the reaction mixture is conveyed into the reaction tube. Further preferably, the starting materials and catalyst are preferably supplied to the method according to the invention in liquid form. For this, it is possible to use relatively high-melting and/or relatively high-viscosity starting materials, for example in the molten state and/or admixed with solvent, for example in the form of a solution, dispersion or emulsion. The catalyst is added to one of the starting materials or else to the starting material mixture prior to entry into the reaction tube. It is also possible to react heterogeneous systems by the process according to the invention, in which case appropriate industrial equipment for conveying the reaction material is required.

The reaction mixture can be fed into the reaction tube either at the end conducted through the inner conductor tube or at the opposite end. The reaction mixture can consequently be conducted through the microwave applicator parallel or anti-parallel to the direction of propagation of the microwaves.

By variation of tube cross section, length of the irradiation zone (this is understood as meaning the length of the reaction tube in which the reaction material is exposed to microwave radiation), flow rate, geometry of the cavity resonator, and the microwave power injected, the reaction conditions are preferably established such that the maximum reaction temperature is achieved as quickly as possible and the residence time at maximum temperature remains sufficiently short that the fewest possible secondary reactions or consecutive reactions occur. To complete the reaction, the reaction material can pass through the reaction tube more than once, optionally after intermediate cooling. In the case of slow reactions, it has often proven useful to keep the reaction product at reaction temperature for a certain time after it leaves the reaction tube. In many cases, it has proven to be useful if the reaction product is cooled immediately after leaving the reaction tube, e.g. by jacket cooling or decompression. It has also proven useful to deactivate the catalyst directly after it has left the reaction tube. This can take place for example by neutralization or, in the case of heterogeneously catalyzed reactions, by filtration.

Preferably, the temperature increase caused by the microwave irradiation is limited to a maximum of 500° C. for example by regulating the microwave intensity, the flow rate and/or by cooling the reaction tube, for example by means of a nitrogen stream. In particular, carrying out the reaction at temperatures between 120° C. and a maximum of 400° C. and specifically between 150° C. and a maximum of 300° C., such as, for example, at temperatures between 180° C. and 270° C., has proven successful.

The duration of the microwave irradiation depends on various factors, such as, for example, the geometry of the reaction tube, the injected microwave energy, the specific reaction and the desired degree of conversion. Usually, the microwave irradiation is undertaken over a period of less than 30 minutes, preferably between 0.01 seconds and 15 minutes, particularly preferably between 0.1 seconds and 10 minutes and in particular between one second and 5 minutes, such as, for example, between 5 seconds and 2 minutes. The intensity (power) of the microwave radiation is adjusted here such that the reaction material has the desired maximum temperature upon leaving the cavity resonator. In a preferred embodiment, the reaction product is cooled as quickly as possible directly after the microwave irradiation is complete to temperatures below 120° C., preferably below 100° C. and especially below 60° C.

Preferably, the reaction is carried out at pressures between 1 bar (atmospheric pressure) and 500 bar, particularly preferably between 1.5 and 200 bar, in particular between 3 bar and 150 bar and especially between 10 bar and 100 bar, such as, for example, between 15 and 50 bar. Working under increased pressure has proven to be particularly useful, which involves working above the boiling temperature (at atmospheric pressure) of the starting materials, products, of any solvent present and/or of the water of reaction formed during the reaction. The pressure is particularly preferably adjusted to a sufficiently high level that the reaction mixture remains in the liquid state and does not boil during the microwave irradiation.

To avoid secondary reactions and to produce the purest possible products, it has proven useful to handle starting materials and products in the presence of an inert protective gas, such as, for example, nitrogen, argon or helium.

Although the starting materials carboxylic acid and alcohol often lead to easy-to-handle reaction mixtures, it has in many cases proven useful to work in the presence of solvents in order, for example, to lower the viscosity of the reaction mixture and/or to fluidize the reaction mixture, especially if it is heterogeneous. For this purpose, it is possible in principle to use all solvents which are inert under the reaction conditions used and do not react with these starting materials and/or the products formed. An important factor when selecting suitable solvents is their polarity, which, on the one hand, determines the dissolution properties and, on the other hand, determines the extent of the interaction with microwave radiation. A particularly important factor when selecting suitable solvents is their dielectric loss ε″. The dielectric loss ε″ describes the proportion of microwave radiation which is converted to heat during the interaction of a substance with microwave radiation. The last-mentioned value has proven to be a particularly important criterion for the suitability of a solvent for carrying out the method according to the invention.

It has proven particularly useful to work in solvents which exhibit the lowest possible microwave absorption and hence make only a small contribution to the heating of the reaction system. Solvents preferred for the method according to the invention have a dielectric loss ε″, measured at room temperature and 2450 MHz, of less than 10 and preferably less than 1, such as, for example, less than 0.5. An overview of the dielectric loss of different solvents can be found, for example, in “Microwave Synthesis” by B. L. Hayes, CEM Publishing 2002. Of suitability for the method according to the invention are in particular solvents with ε″ values below 10, such as N-methylpyrrolidone, N,N-dimethylformamide or acetone, and in particular solvents with ε″ values below 1. Examples of particularly preferred solvents with ε″ values below 1 are aromatic and/or aliphatic hydrocarbons, such as, for example, toluene, xylene, ethylbenzene, tetralin, hexane, cyclohexane, decane, pentadecane, decalin, and also commercial hydrocarbon mixtures, such as benzine fractions, kerosene, solvent naphtha, Shellsol® AB, Solvesso® 150, Solvesso® 200, Exxsol®, Isopar® and Shellsol® grades. Solvent mixtures which have ε″ values preferably below 10 and specifically below 1 are equally preferred for carrying out the method according to the invention.

In a further preferred embodiment, the method according to the invention is carried out in solvents with higher ε″ values of, for example 5 or higher, such as in particular with ε″ values of 10 and higher. This embodiment has proven to be useful particularly in the case of the reaction of reaction mixtures which themselves, i.e. without the presence of solvents and/or diluents, exhibit only a very low microwave absorption. Thus, this embodiment has proven to be particularly useful in the case of reaction mixtures which have a dielectric loss ε″ of less than 10 and preferably less than 1. However, the accelerated heating of the reaction mixture often observed as a result of the solvent addition requires measures for maintaining the maximum temperature.

When working in the presence of solvents, their proportion in the reaction mixture is preferably between 1 and 95% by weight, particularly preferably between 2 and 90% by weight, specifically between 5 and 85% by weight and in particular between 10 and 75% by weight, such as, for example, between 30 and 60% by weight. The reaction is particularly preferably carried out without a solvent.

In a further preferred embodiment, substances are added to the reaction mixture that are insoluble in said mixture and absorb microwaves to a large extent. These lead to a considerable local heating of the reaction mixture and consequently to further accelerated reactions. One suitable heat collector of this type is, for example, graphite.

Microwaves is the term used to refer to electromagnetic rays with a wavelength between about 1 cm and 1 m and frequencies between about 300 MHz and 30 GHz. This frequency range is suitable in principle for the method according to the invention. For the method according to the invention, preference is given to using microwave radiation with the frequencies approved for industrial, scientific, medical, domestic or similar applications, such as, for example, with frequencies of 915 MHz, 2.45 GHz, 5.8 GHz or 24.12 GHz.

The microwave power to be injected into the cavity resonator for carrying out the method according to the invention is in particular dependent on the desired reaction temperature, but also on the geometry of the reaction tube and hence the reaction volume, and also the flow rate of the reaction material through the heating zone. It is usually between 200 W and several 100 kW and in particular between 500 W and 100 kW, such as, for example, between 1 kW and 70 kW. It can be generated by means of one or more microwave generators.

In a preferred embodiment, the reaction is carried out in a pressure-resistant, chemically inert tube, where the water of reaction which is formed, and possibly starting materials and, if present, solvent, lead to a buildup in pressure. When the reaction is complete, the overpressure can be used by means of decompression for volatalization and removal of water of reaction, excess starting materials, and optionally solvent and/or for cooling the reaction product. In a further embodiment, the water of reaction formed is, after cooling and/or decompression, separated off by customary methods such as, for example, phase separation, distillation, stripping, flashing and/or absorption.

To achieve particularly high degrees of conversion, it has in many cases proven useful to expose the resulting reaction mixture, following removal of water of reaction and also, if appropriate, discharge of product and/or by-product, again to microwave irradiation, in which case the ratio of the reactants used may have to be supplemented to compensate for consumed or deficient starting materials.

The advantages of the method according to the invention are a very uniform irradiation of the reaction material in the center of a symmetric microwave field within a reaction tube, the longitudinal axis of which is in the direction of propagation of the microwaves of a monomode microwave applicator and in particular within a E₀₁ cavity resonator for example with coaxial transition. Here, the reactor design according to the invention also allows reactions to be carried out at very high pressures and/or temperatures. As a result of increasing the temperature and/or pressure, a significant increase in the degree of conversion and yield is observed even compared with known microwave reactors, without resulting in undesired secondary reactions and/or discolorations. Surprisingly, a very high efficiency in the utilization of the microwave energy is achieved here while utilizing the microwave energy injected into the cavity resonator, said efficiency usually being more than 50%, often more than 80%, sometimes more than 90% and in specific cases above 95%, such as, for example, above 98%, of the injected microwave power and hence offers economical and also ecological advantages over conventional production methods and also over microwave methods in the prior art.

Moreover, the method according to the invention allows a controlled, safe and reproducible reaction regime. Since the reaction material is moved in the reaction tube parallel to the direction of propagation of the microwaves, known overheating phenomena as a result of uncontrolled field distributions, which lead to local overheating as a result of changing intensities of the microwave field, for example in wave crests and nodes, are balanced out by the flowing motion of the reaction mixture. The advantages mentioned also make it possible to work with high microwave powers of more than 1 kW, such as, for example, 2 to 10 kW and in particular 5 to 100 kW, and sometimes even higher, and hence, in combination with only a short residence time in the cavity resonator, to accomplish large production quantities of 100 and more tons per year in one plant.

In this connection, it was surprising that, in spite of the only very short residence time of the reaction mixture in the microwave field in the flow tube with continuous flow, very substantial esterification takes place with conversions generally of more than 80%, often even more than 90%, such as, for example, more than 95%, based on the component used in deficit, without the formation of noteworthy amounts of by-products. Furthermore, it was surprising that the stated conversions can be achieved under these reaction conditions without separating off the water of reaction formed during the esterification. In the case of a corresponding reaction of these reaction mixtures in a flow tube with identical dimensions and with thermal jacket heating, to achieve suitable reaction temperatures, extremely high wall temperatures are required, which led to the formation of undefined polymers and colored species, but bring about significantly lower ester formation in the same time interval. Furthermore, the products produced by the method according to the invention have very low metal contents, without requiring further work-up of the crude products. For example, the metal contents of the products produced by the method according to the invention, based on iron as the main element, are usually below 25 ppm, preferably below 15 ppm, specifically below 10 ppm, such as, for example, between 0.01 and 5 ppm, of iron.

The method according to the invention thus allows a very rapid, energy-saving and cost-effective production of carboxylic acid esters in high yields and with high purity in industrial-scale amounts. Besides the water of reaction, this method does not produce any significant amounts of by-products. Such rapid and selective reactions cannot be achieved by conventional methods and were not to be expected solely as a result of heating to high temperatures.

### EXAMPLES

The reactions of the reaction mixtures under microwave irradiation were carried out in a ceramic tube (60×1 cm) which was located in axial symmetry in a cylindrical cavity resonator (60×10 cm). On one of the ends of the cavity resonator, the ceramic tube passed through the cavity of an inner conductor tube functioning as coupling antenna. The microwave field with a frequency of 2.45 GHz, produced by a magnetron, was coupled into the cavity resonator by means of the coupling antenna (E₀₁ cavity applicator; monomode), in which a stationary wave was formed.

The microwave power was in each case adjusted via the experiment time in such a way that the desired temperature of the reaction material at the end of the irradiation zone was kept constant. The microwave powers specified in the experiment descriptions therefore represent the mean value of the injected microwave power over time. The measurement of the temperature of the reaction mixture was undertaken directly after it had left the irradiation zone (distance of about 15 cm in an insulated stainless steel capillary, Ø 1 cm) by means of a Pt100 temperature sensor. Microwave energy not absorbed directly by the reaction mixture was reflected at the end of the cavity resonator positioned at the opposite end to the coupling antenna; the microwave energy which was also not absorbed by the reaction mixture on the return path and reflected back in the direction of the magnetron was passed with the aid of a prism system (circulator) into a water-containing vessel. The difference between energy injected and heating of this water load was used to calculate the microwave energy introduced into the reaction material.

By means of a high-pressure pump and of a suitable pressure-release valve, the reaction mixture in the reaction tube was placed under a operating pressure which sufficed to always keep all of the starting materials and products or condensation products in the liquid state. The reaction mixtures produced from carboxylic acid and alcohol were pumped at a constant flow rate through the reaction tube, and the residence time in the irradiation zone was adjusted by modifying the flow rate.

The products were analyzed by means of ¹H-NMR spectroscopy at 500 MHz in CDCl₃. The properties were determined by means of atomic absorption spectroscopy.

### Example 1

### Production of Butyl Acetate

In a 10 l Büchi stirred autoclave with stirrer, internal thermometer and pressure equalizer, 3.56 kg of n-butanol (48 mol) were introduced as initial charge and admixed with 1.44 kg of acetic acid (24 mol) and 0.05 kg of methanesulfonic acid.

The mixture obtained in this way was pumped through the reaction tube continuously at 5 l/h at an operating pressure of 25 bar and exposed to a microwave power of 2.7 kW, 91% of which was absorbed by the reaction material. The residence time of the reaction mixture in the irradiation zone was ca. 35 seconds. At the end of the reaction tube, the reaction mixture had a temperature of 275° C. The reaction mixture was cooled to room temperature directly after leaving the reactor using a high-intensity heat exchanger and admixed with hydrogencarbonate solution to neutralize the catalyst.

A conversion of 84% of theory was achieved. The reaction product was virtually colorless and comprised <2 ppm of iron. Following distillative removal of water of reaction and unreacted starting materials and distillation of the product, 2.25 kg of butyl acetate with a purity of >99% were obtained by means of vacuum distillation.

### Example 2

### Production of Methyl Hexanoate

In a 10 l Büchi stirred autoclave with stirrer, internal thermometer and pressure equalizer, 4.94 kg of hexanoic acid (43 mol) were introduced as initial charge and admixed with 2.56 kg of methanol (80 mol) and 0.075 kg of methanesulfonic acid.

The mixture obtained in this way was pumped through the reaction tube continuously at 7.5 l/h at an operating pressure of 35 bar and exposed to a microwave power of 3.0 kW, 90% of which was absorbed by the reaction material. The residence time of the reaction mixture in the irradiation zone was ca. 23 seconds. At the end of the reaction tube, the reaction mixture had a temperature of 279° C.

The reaction mixture was cooled to room temperature directly after leaving the reactor using a high-intensity heat exchanger. Following neutralization of the catalyst with hydrogencarbonate solution, phase separation and distillative removal of residual water and excess methanol, 5.09 kg of methyl hexanoate (91% of theory) with a residual acid number of 0.5 mg KOH/g were obtained. The reaction product was slightly yellowish in color, the iron content of the product was below 3 ppm.

### Example 3

### Production of Methyl Methacryl

In a 10 l Büchi stirred autoclave with stirrer, internal thermometer and pressure equalizer, 4.8 kg of methacrylic acid (56 mol) were introduced as initial charge and admixed with 2.7 kg of methanol (84 mol), 1.5 g of phenothiazine (inhibitor) and 0.075 kg of methanesulfonic acid.

The mixture obtained in this way was pumped continuously through the reaction tube at 7.5 l/h at an operating pressure of 35 bar and exposed to a microwave power of 3.6 kW, 95% of which was absorbed by the reaction material. The residence time of the reaction mixture in the irradiation zone was ca. 23 seconds. At the end of the reaction tube, the reaction mixture had a temperature of 249° C. The reaction mixture was cooled to room temperature directly after leaving the reactor using a high-intensity heat exchanger.

A conversion of 82% of theory was achieved. The reaction product was slightly yellowish in color. Following neutralization of the catalyst with hydrogencarbonate solution, distillative removal of water of reaction and unreacted starting materials and distillation of the product, 4.32 kg of methyl methacrylate with a purity of >99% were obtained.

### Example 4

### Production of Stearyl Acrylate

In a 10 l Büchi stirred autoclave with stirrer, internal thermometer and pressure equalizer, 3.6 kg of acrylic acid (50 mol) were introduced as initial charge and admixed with 6.8 kg of stearyl alcohol (25 mol), 3 g of phenothiazine (inhibitor).

The mixture obtained in this way was pumped continuously through the reaction tube at 4 l/h at an operating pressure of 27 bar and subjected to a microwave power of 3.1 kW, 90% of which was absorbed by the reaction material. The residence time of the reaction mixture in the irradiation zone was ca. 43 seconds. At the end of the reaction tube, the reaction mixture had a temperature of 254° C. The reaction mixture was cooled to 60° C. directly after leaving the reactor using a high-intensity heat exchanger.

A conversion of 93% of theory was achieved. The reaction product was yellowish in color. Following distillative removal of excess acrylic acid, 7.34 kg of stearyl acrylate with a purity of >97% were obtained.

### Example 5

### Production of Undecyl 2-Hydroxypropanoate

In a 10 l Büchi stirred autoclave with stirrer, internal thermometer and pressure equalizer, 2.25 kg of lactic acid (as 90% strength aqueous solution, 22.5 mol) were introduced as initial charge and admixed with 7.75 kg of undecyl alcohol (Exxal® 11 from Exxon, 45 mol) and 0.075 kg of methanesulfonic acid.

The mixture obtained in this way was pumped continuously through the reaction tube at 6 l/h at an operating pressure of 25 bar and exposed to a microwave power of 3.7 kW, 92% of which was absorbed by the reaction material. The residence time of the reaction mixture in the irradiation zone was ca. 29 seconds. At the end of the reaction tube, the reaction mixture had a temperature of 267° C. The reaction mixture was cooled to room temperature directly after leaving the reactor using a high-intensity heat exchanger.

A conversion of 89% of theory was achieved. The reaction product was colorless. Following neutralization of the catalyst with hydrogencarbonate solution and distillative removal of water of reaction and unreacted starting materials, 4.7 kg of undecyl lactate with a purity of >98.5% were obtained after vacuum distillation at 1 mbar and 170° C.

### Example 6

### Production of 2-Ethylhexyl 2-Hydroxypropanoate

In a 10 l Büchi stirred autoclave with stirrer, internal thermometer and pressure equalizer, 2.5 kg of lactic acid (as 90% strength aqueous solution, 25 mol) were introduced as initial charge and admixed with 6.5 kg of 2-ethylhexanol (50 mol) and also 0.075 kg of methanesulfonic acid.

The mixture obtained in this way was pumped through the reaction tube continuously at 6 l/h at an operating pressure of 25 bar and exposed to a microwave power of 3.2 kW, 94% of which was absorbed by the reaction material. The residence time of the reaction mixture in the irradiation zone was ca. 29 seconds. At the end of the reaction tube, the reaction mixture had a temperature of 271° C. The reaction mixture was cooled to room temperature directly after leaving the reactor using a high-intensity heat exchanger.

A conversion of 92% of theory was achieved. The reaction product was colorless. Following neutralization of the catalyst with hydrogencarbonate solution and distillative removal of water of reaction and unreacted starting materials, 4.52 kg of 2-ethylhexyl lactate with a purity of >99% were obtained after vacuum distillation.

### Example 7

### Production of Poly-(2-Hydroxypropanoic Acid)

In a 10 l Büchi stirred autoclave with stirrer, internal thermometer and pressure equalizer, 5.0 kg of lactic acid (as 90% strength aqueous solution, 50 mol) were introduced as initial charge, admixed with 10 g of conc. sulfuric acid (0.2% by weight) and heated to 60° C. The lactic acid solution was pumped through the reaction tube continuously at 3.5 l/h at an operating pressure of 25 bar and exposed to a microwave power of 3.4 kW, 92% of which was absorbed by the reaction material. The residence time of the reaction mixture in the irradiation zone was ca. 50 seconds. At the end of the reaction tube, the reaction mixture had a temperature of 235° C. The reaction mixture was cooled to room temperature directly after leaving the reactor using a high-intensity heat exchanger.

A conversion, based on the COOH functionalities used, of 72% of theory was achieved (measured by means of acid number titration), which corresponds to an average degree of polymerization of approximately 4. The reaction product was colorless to slightly yellowish and distinctly viscous.

1. A continuous method for producing an aliphatic carboxylic ester, in which at least one aliphatic carboxylic acid of the formula (I)
R¹—COOH (I)
in which R¹ is hydrogen or an optionally substituted aliphatic hydrocarbon radical having 1 to 50 carbon atoms,
is reacted with at least one alcohol of the formula (II)
R²—(OH)ₙ (II)
in which
R² is an optionally substituted hydrocarbon radical having 1 to 100 carbon atoms and
n is a number from 1 to 10,
in the presence of at least one esterification catalyst with microwave irradiation in a reaction tube, the longitudinal axis of which extends in the direction of propagation of the microwaves of a monomode microwave applicator, to give the ester, in which the irradiation of the reaction mixture takes place with microwaves in a largely microwave-transparent reaction tube within a hollow conductor connected via waveguides to a microwave generator.

2. A method as claimed in claim 1, in which the microwave applicator is designed as a cavity resonator.

3. A method as claimed in claim 1, in which the microwave applicator is configured as a cavity resonator of the reflection type.

4. A method as claimed in claim 1, in which the reaction tube is aligned axially with a central axis of symmetry of the hollow conductor.

5. A method as claimed in claim 2, in which the irradiation of the reaction mixture takes place in a cavity resonator with a coaxial transition of the microwaves.

6. A method as claimed in claim 2, in which the cavity resonator is operated in the E₀₁ₙ mode, where n is an integer from 1 to 200.

7. A method as claimed in claim 2, in which a stationary wave is formed in the cavity resonator.

8. A method as claimed in claim 1, in which the reaction material is heated by the microwave irradiation to temperatures between 120 and 500° C.

9. A method as claimed claim 1, in which the microwave irradiation takes place at pressures above atmospheric pressure.

10. A method as claimed in claim 1, in which R¹ is an optionally substituted aliphatic hydrocarbon radical having 2 to 30 carbon atoms.

11. A method as claimed in claim 1, in which R¹ is an optionally substituted saturated alkyl radical having 1, 2, 3 or 4 carbon atoms.

12. A method as claimed in claim 1, in which R¹ is an optionally substituted alkenyl group having 2 to 4 carbon atoms.

13. A method as claimed in claim 1, in which R¹ carries at least one further substituent selected from the group consisting of a carboxyl group, a hydroxyl group and a C₅-C₂₀-aryl group.

14. A method as claimed in claim 1, in which R¹ is an optionally substituted aliphatic hydrocarbon radical having 5 to 50 carbon atoms.

15. A method as claimed in claim 1, in which R² is an optionally substituted aliphatic radical having 2 to 24 carbon atoms.

16. A method as claimed in claim 1, in which R² is an optionally substituted C₆-C₁₂-aryl group or an optionally substituted heteroaromatic group having 5 to 12 ring members.

17. A method as claimed in claim 1, in which R² carries one, two, three, four, five or six OH groups.

18. A method as claimed in claim 1, in which R² is radicals of the formula (III)
—(R⁴—O)ⁿ—R⁵ (III)
in which
R⁴ is an alkylene group having 2 to 18 carbon atoms or mixtures thereof,
R⁵ is hydrogen or a hydrocarbon radical having 1 to 24 carbon atoms or a group of the formula —R⁴—NR¹⁰R¹¹,
n is a number between 1 and 500, and
R¹⁰, R¹¹ independently of one another, are an aliphatic radical having 1 to 24 carbon atoms, an aryl group or heteroaryl group having 5 to 12 ring members, a poly(oxyalkylene) group having 1 to 50 poly(oxyalkylene) units, where the polyoxyalkylene units are derived from alkylene oxide units having 2 to 6 carbon atoms, or R¹⁰ and R¹¹ together with the nitrogen atom to which they are bonded form a ring having 4, 5, 6 or more ring members.

19. A method as claimed in claim 1, in which R¹ is a hydroxyl group and R² is a carboxyl group.

20. A method as claimed in claim 19, in which R¹ and R² are the same.

21. A method as claimed in claim 1, in which aliphatic carboxylic acid (I) and alcohol (II) are reacted in the molar ratio from 20:1 to 1:20, in each case based on the mole equivalents of carboxyl and hydroxyl groups.

22. A method as claimed in claim 1, which is carried out in the presence of homogeneous catalysts, heterogeneous catalysts or mixtures thereof.
